(19) United States
(12) Patent Application Publication
(10) Pub. No.: US 2019/0380681 A1
(43) Pub. Date: Dec. 19, 2019

(54) METHOD AND SYSTEMS FOR A PORTABLE ULTRASOUND IMAGING SYSTEM

(71) Applicant: General Electric Company, Schenectady, NY (US)

(72) Inventors: Robert Andrew Meurer, Waukesha, WI (US); Ross Christopher Stalter, Hartland, WI (US); Craig Robert Loomis, Shorewood, WI (US); Shabtay Eliad, Zichron Yaakov (IL); William Zang, Grafton, WI (US); Del Jefferson France, San Rafael, CA (US)

(21) Appl. No.: 16/009,096

(22) Filed: Jun. 14, 2018

(51) Int. Cl. A61B 8/00

(52) U.S. Cl. CPC A61B 8/4427; A61B 8/54; A61B 8/467; A61B 8/461

(57) ABSTRACT

Various methods and systems are provided for portable ultrasound imaging. In one example, a portable ultrasound imaging system includes: a touch-sensitive graphical display; a controller in electronic communication with the display; and a housing enclosing the display and the controller, the housing including: an electronic input connection adapted to electrically couple the controller with an ultrasound probe; a probe storage section having an opening shaped to receive the ultrasound probe; and a first mating feature adapted to engage with a counterpart, second mating feature of a cradle of a support stand to removably couple the housing to the cradle.

### FIELD

Embodiments of the subject matter disclosed herein relate to diagnostic medical imaging, and more particularly, to ultrasound imaging.

### BACKGROUND

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device. In one example, the display device may be a touch-sensitive display, also referred to as a touchscreen. A user may interact with the touchscreen to analyze the displayed image. For example, a user may use their fingers on the touchscreen to position a region of interest (ROI), place measurement calipers, or the like.

### BRIEF DESCRIPTION

In one embodiment, a portable ultrasound imaging system comprises: a touch-sensitive graphical display; a controller in electronic communication with the display; and a housing enclosing the display and the controller, the housing including: an electronic input connection adapted to electrically couple the controller with an ultrasound probe; a probe storage section having a first opening shaped to receive the ultrasound probe; and a first mating feature adapted to engage with a counterpart, second mating feature of a cradle of a support stand to removably couple the housing to the cradle.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system, according to an embodiment of the invention.

FIG. 2 shows a front perspective view of an ultrasound imaging assembly including a portable ultrasound system.

FIG. 3 shows a rear perspective view of the ultrasound imaging assembly of FIG. 2.

FIG. 4 shows a front perspective view of the ultrasound imaging assembly of FIGS. 2-3, with a support stand of the ultrasound imaging assembly in a lowered position.

FIG. 5 shows a top view of a tray coupled to the ultrasound imaging assembly of FIGS. 2-4.

FIG. 6 shows a front perspective view of a cradle of the ultrasound imaging assembly of FIGS. 2-5.

FIG. 7 shows a top view of the cradle of FIG. 6.

FIG. 8 shows a rear perspective view of the cradle of FIGS. 6-7.

FIG. 9 shows a front perspective view of the portable ultrasound system of the ultrasound imaging assembly of FIGS. 2-5.

FIG. 10 shows a rear perspective view of the portable ultrasound system of FIG. 9.

FIG. 11 shows a top view of the portable ultrasound system of FIGS. 9-10.

FIG. 12 shows a bottom view of the portable ultrasound system of FIGS. 9-11.

FIG. 13 shows an exploded view of an accessory support of the ultrasound imaging assembly of FIGS. 2-5.

FIG. 14 shows an assembled view of the accessory support of FIG. 13.

FIG. 15 shows an exploded view of a support arm of the portable ultrasound system of FIGS. 9-12.

FIG. 16 shows a side view of the portable ultrasound system of FIGS. 9-12 and 13, with the portable ultrasound system positioned on a support surface, and with the support arm of FIG. 15 in a first extended position.

FIG. 17 shows a side view of the portable ultrasound system of FIGS. 9-12 and 13-14, with the portable ultrasound system positioned on a support surface, and with the support arm of FIGS. 15-16 in a second extended position.

FIG. 18 shows a side perspective view of a cable support coupled to the portable ultrasound system of FIGS. 9-12 and 13-14.

FIG. 19 shows a first perspective view of the cable support of FIG. 18 removed from the portable ultrasound system.

FIG. 20 shows a second perspective view of the cable support of FIG. 19 with the cable support in a decompressed state.

FIG. 21 shows the cable support in a compressed state.

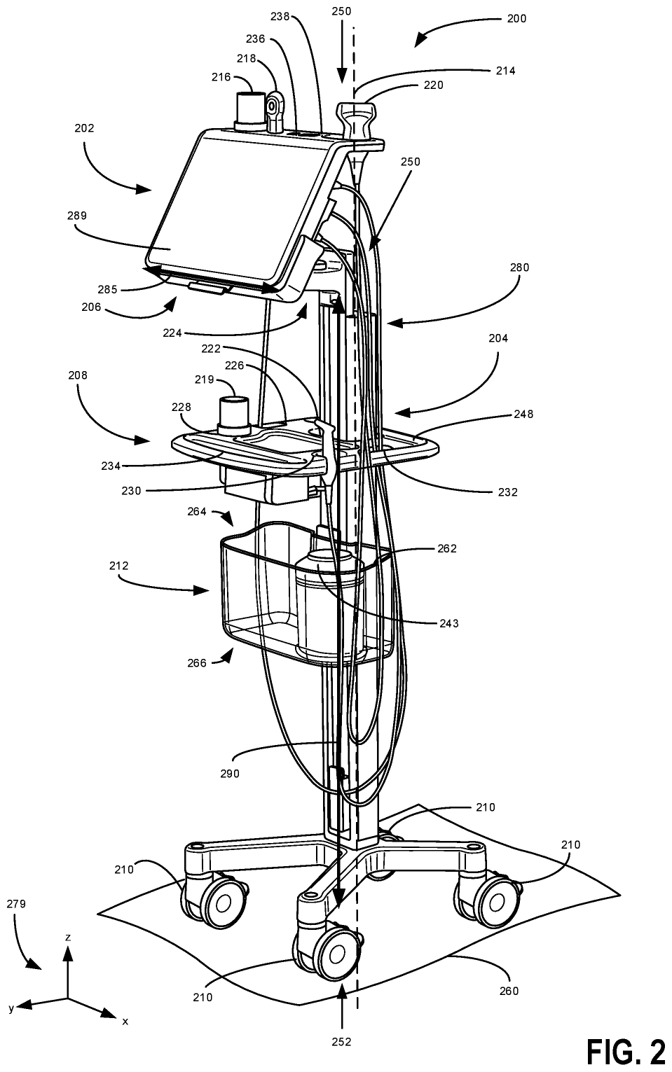

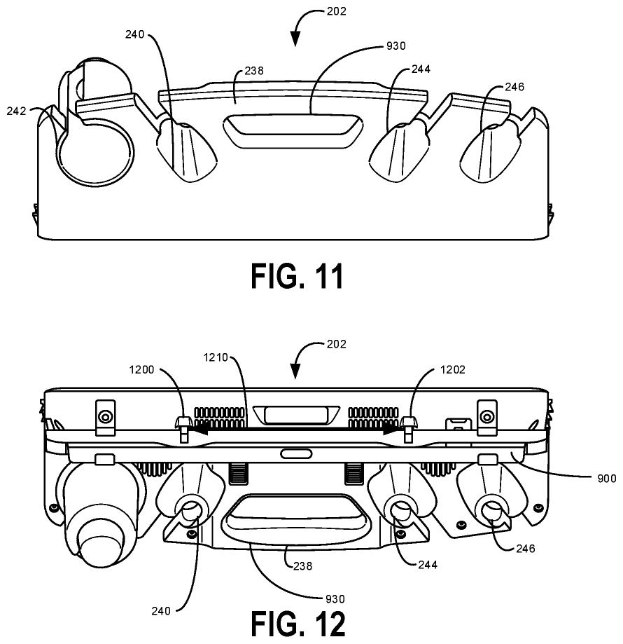

FIGS. 2-21 are shown to scale, though other relative dimensions may be used, if desired.

### DETAILED DESCRIPTION

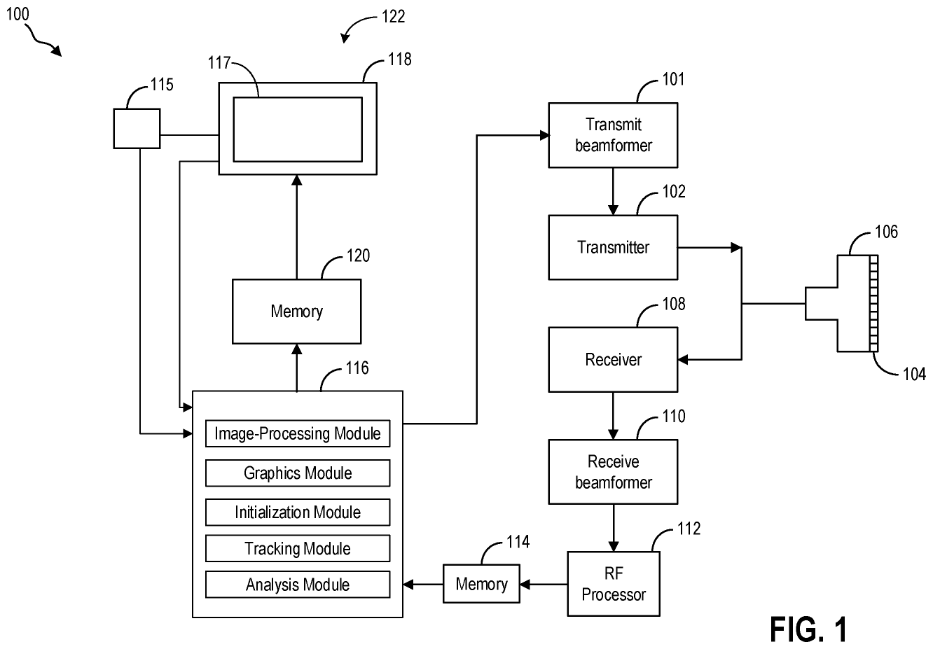

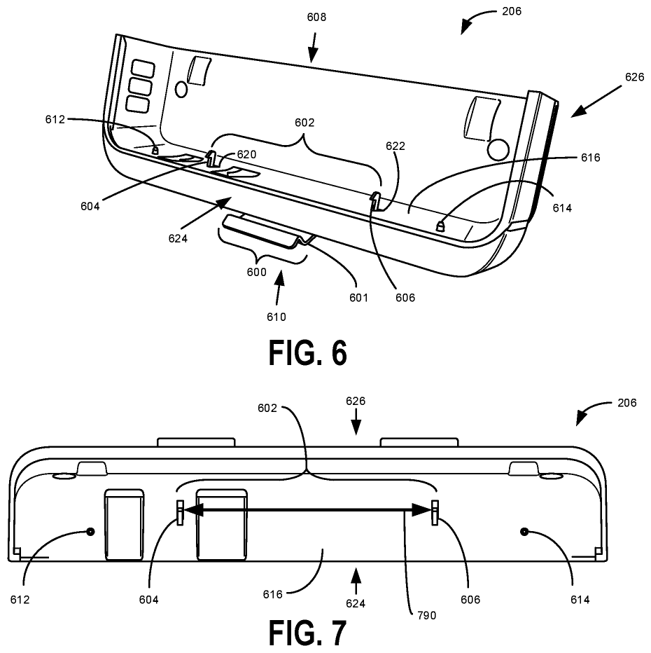

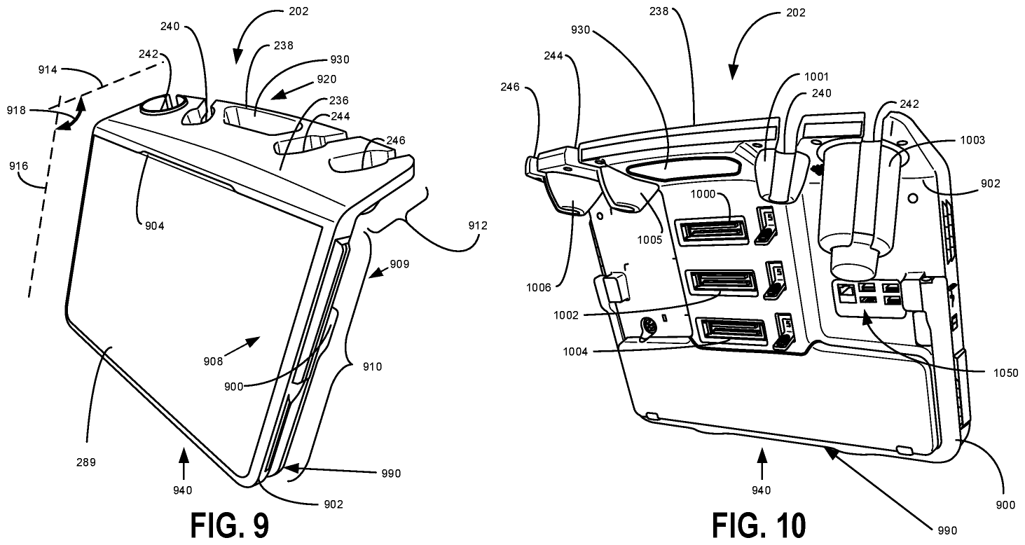

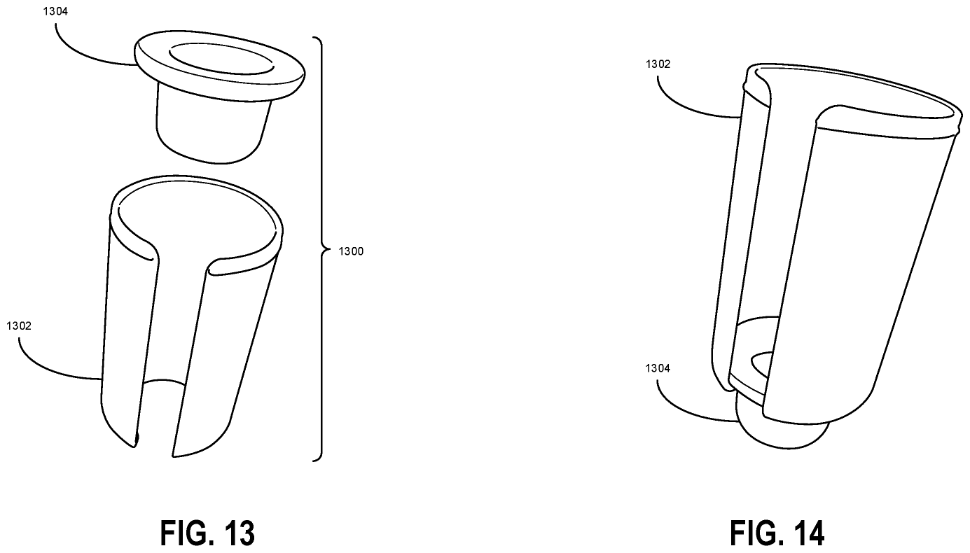

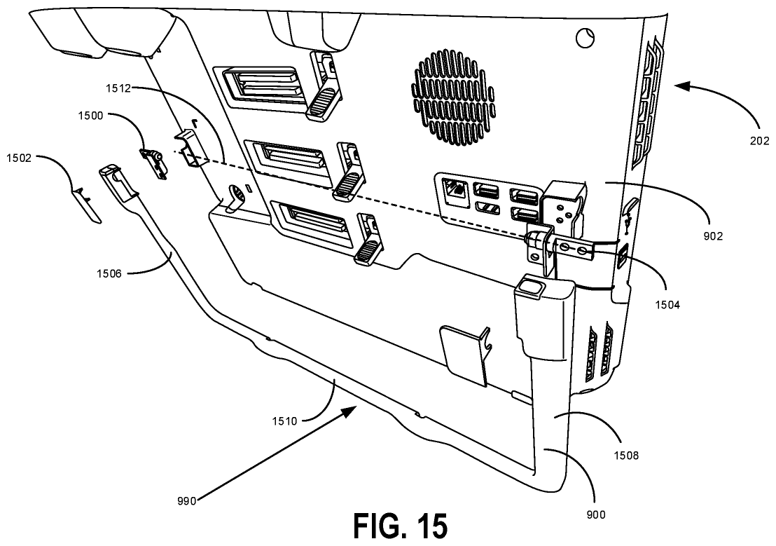

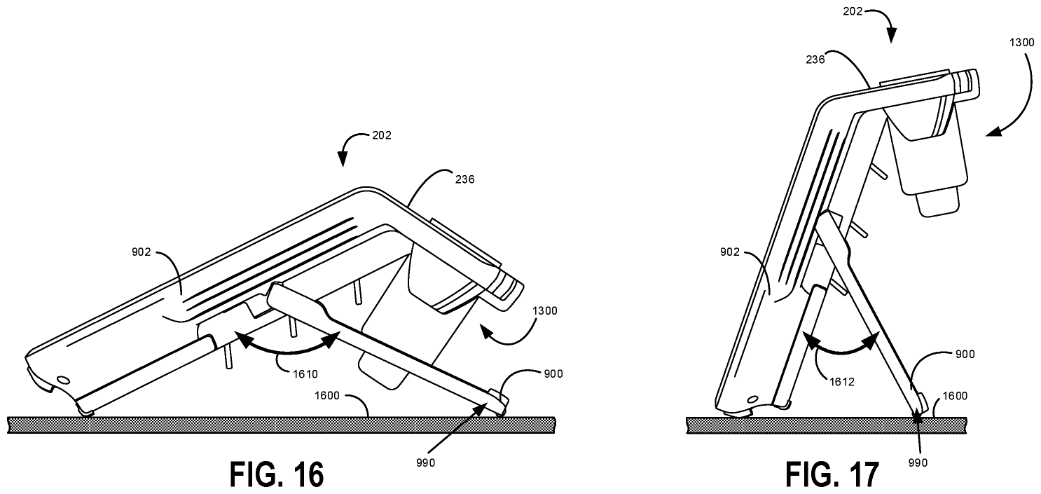

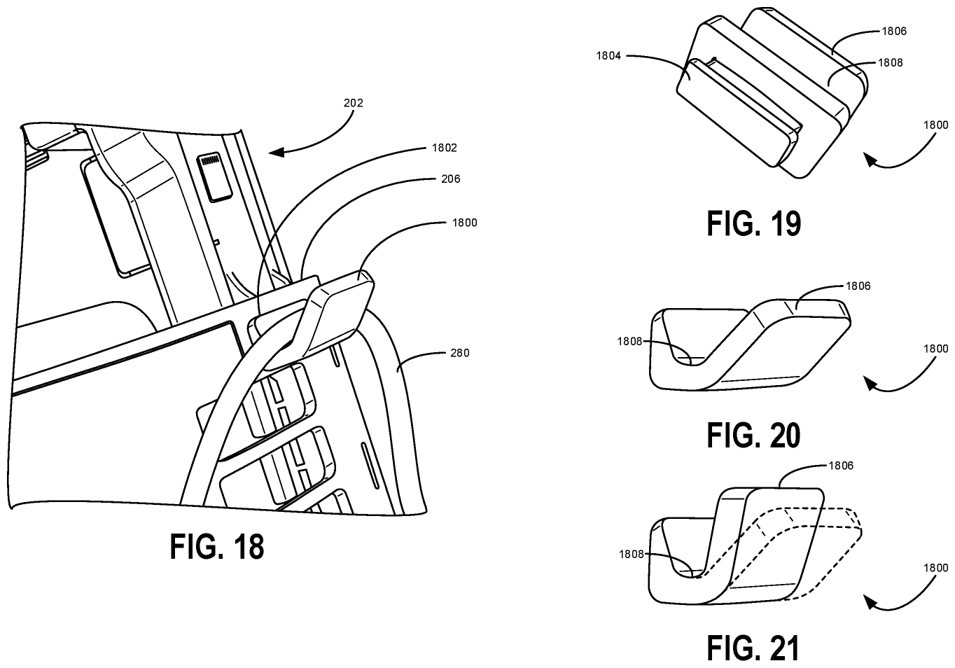

The following description relates to various embodiments of an ultrasound imaging system, such as the ultrasound imaging system shown in FIG. 1. The ultrasound imaging system is a portable ultrasound imaging system that may be included within an ultrasound imaging assembly, such as the ultrasound imaging assembly shown by FIGS. 2-5. The ultrasound imaging assembly includes a support stand and a pivotable cradle, such as the cradle shown by FIGS. 6-8, with the portable ultrasound system configured to removably couple with the cradle. The portable ultrasound system includes a touchscreen display and a plurality of openings adapted to maintain a position of one or more probes, accessories, etc. relative to the portable ultrasound system, as shown by FIGS. 9-12. One or more of the openings may include a sleeve adapted to support the probes and/or accessories, such as the sleeve shown by FIGS. 13-14. Further, the portable ultrasound system includes a support stand, such as the support stand shown by FIG. 15, pivotable to a plurality of extended positions, such as the extended positions shown by FIGS. 16-17. The support stand may maintain a position of the portable ultrasound system on surfaces other than the cradle, such as a table. Further, the portable ultrasound system may include cable management features, such as the cable support tab shown by FIGS. 18-21. In this way, a portability of the portable ultrasound system is increased, and an operator of the portable ultrasound system may more easily move the portable ultrasound system from the cradle to a different location or vice versa.

FIG. 1 illustrates a block diagram of a system 100 according to one embodiment. In the illustrated embodiment, the system 100 is an imaging system and, more specifically, an ultrasound imaging system. As shown, the system 100 includes multiple components. The components may be coupled to one another to form a single structure. In the examples described herein, system 100 is a unitary system that is capable of being moved (e.g., portably) from room to room. For example, the system 100 may include one or more components configured to couple the system 100 to a wheeled cart, similar to the system described below with reference to FIGS. 2-17.

In the illustrated embodiment, the system 100 includes a transmit beamformer 101 and transmitter 102 that drives an array of elements 104 (e.g., piezoelectric crystals) within a diagnostic ultrasound probe 106 (or transducer) to emit pulsed ultrasonic signals into a body or volume (not shown) of a subject. The elements 104 and the probe 106 may have a variety of geometries. The ultrasonic signals are back-scattered from structures in the body, for example, blood vessels and surrounding tissue, to produce echoes that return to the elements 104. The echoes are received by a receiver 108. The received echoes are provided to a receive beamformer 110 that performs beamforming and outputs an RF signal. The RF signal is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage).

The system 100 also includes a system controller 116 that includes a plurality of modules, which may be part of a single processing unit (e.g., processor) or distributed across multiple processing units. The system controller 116 is configured to control operation of the system 100. For example, the system controller 116 may include an image-processing module that receives image data (e.g., ultrasound signals in the form of RF signal data or IQ data pairs) and processes image data. For example, the image-processing module may process the ultrasound signals to generate slices or frames of ultrasound information (e.g., ultrasound images) for displaying to the operator. The image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D) or three-dimensional (3D). When multiple two-dimensional (2D) images are obtained, the image-processing module may also be configured to stabilize or register the images.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices of acquired ultrasound information that are not scheduled to be displayed immediately. The image memory 120 may comprise any known data storage medium, for example, a permanent storage medium, removable storage medium, and the like. Additionally, the image memory 120 may be a non-transitory storage medium.

In operation, an ultrasound system may acquire data, for example, volumetric data sets by various techniques (for example, 3D scanning, real-time 3D imaging, volume scanning, 2D scanning with probes having positioning sensors, freehand scanning using a voxel correlation technique, scanning using 2D or matrix array probes, and the like). Ultrasound images of the system 100 may be generated from the acquired data (at the controller 116) and displayed to the operator or user on the display device 118.

The system controller 116 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In the examples described herein, the display device 118 is a touchscreen display that enables the operator to adjust operating parameters of the system 100 by directly interacting with (e.g., touching) the display device 118. For example, the display device 118 may be configured such that when a user moves a finger/glove/stylus across the face of the display device 118, a cursor atop the ultrasound image on the display area 117 moves in a corresponding manner The display device 118 may detect a presence of a touch from the operator on the display area 117 and may also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information from the controller 116 to the operator by displaying the information to the operator. The display device 118 and/or the user interface 122 may also communicative audibly. The display device 118 is configured to present information to the operator during the imaging session. The information presented may include ultrasound images, graphical elements, user-selectable elements, and other information (e.g., administrative information, personal information of the patient, and the like). In some embodiments, the user interface 122 may be additionally configured to interface with (e.g., electronically couple to) one or more user interface input devices 115, such as a physical keyboard, mouse, and/or touchpad.

In addition to the image-processing module, the system controller 116 may also include a graphics module, an initialization module, a tracking module, and an analysis module. The image-processing module, the graphics module, the initialization module, the tracking module, and the analysis module may coordinate with one another to present information to the operator during and/or after the imaging session. For example, the image-processing module may be configured to display an acquired image on the display device 118, and the graphics module may be configured to display designated graphics along with the ultrasound image, such as graphical outlines, which represent lumens or vessel walls in the acquired image. The image-processing and/or graphics modules within the system controller 116, may also be configured to generate a 3D rendering or image (not shown) of the entire vascular structure.

In some embodiments, the system controller 116 may also house an image-recognition module (not shown), which accesses stored images/videos (i.e., an image library) from either or both of the memory 114 and the memory 120, before analyzing them. For example, knowing the parameters under which a protocol is being carried out (ultrasound type, scan plane, tissue being imaged, etc.) the image recognition module may compare a live image on the display area 117, to one stored in memory 120, in order to analyze the image and thereby improve the accuracy of placing and utilizing analytical tools.. In an alternative embodiment, instead of utilizing an image recognition module and image library, the system controller may house instructions for analyzing acquired imaging data (e.g., ultrasound images/videos acquired with the probe) and automatically determining a desired placement of one or more analytical tools, such as a region of interest.

The screen of the display area 117 of the display device 118 is made up of a series of pixels which display the data acquired with the probe 106. The acquired data includes one or more imaging parameters calculated for each pixel, or group of pixels (for example, a group of pixels assigned the same parameter value), of the display, where the one or more calculated image parameters includes one or more of an intensity, velocity, color flow velocity, texture, graininess, contractility, deformation, and rate of deformation value. The series of pixels then make up the displayed image generated from the acquired ultrasound data.

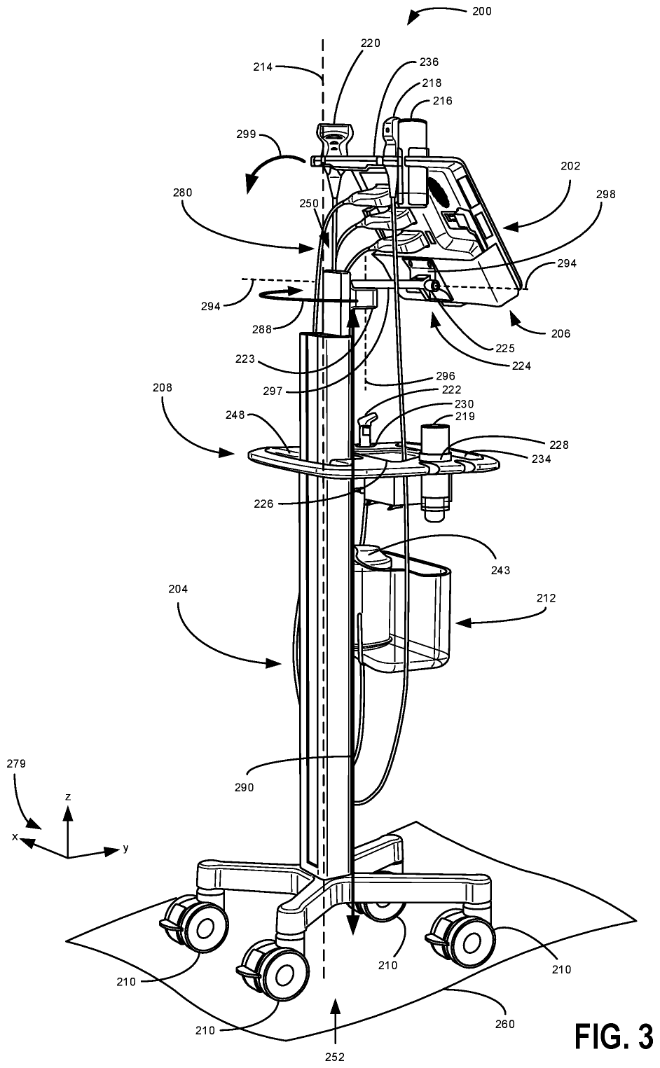

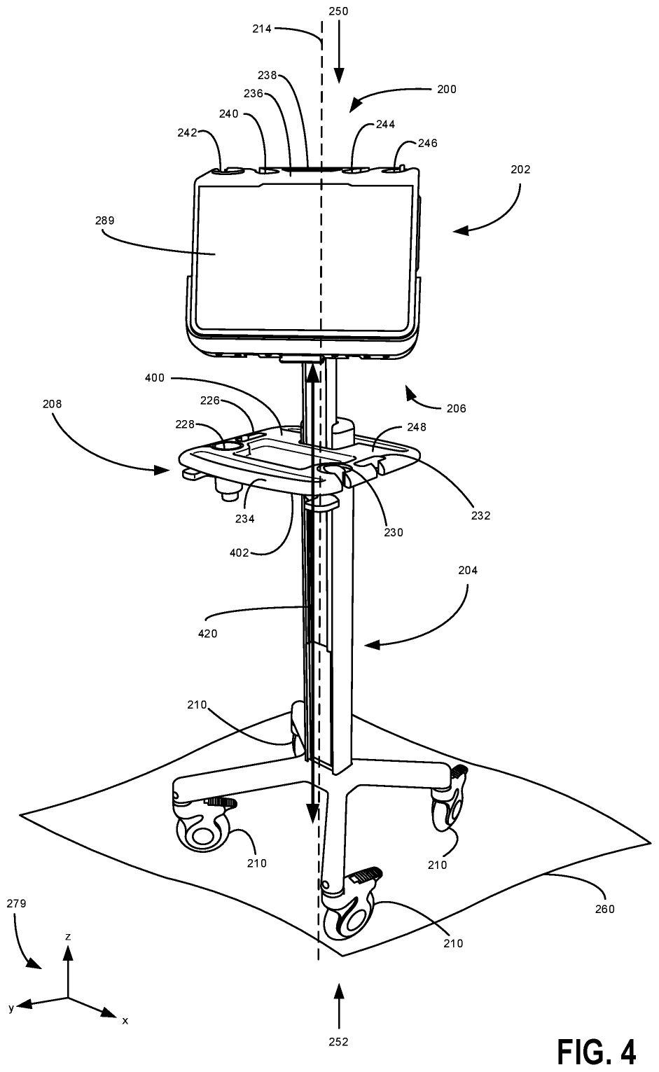

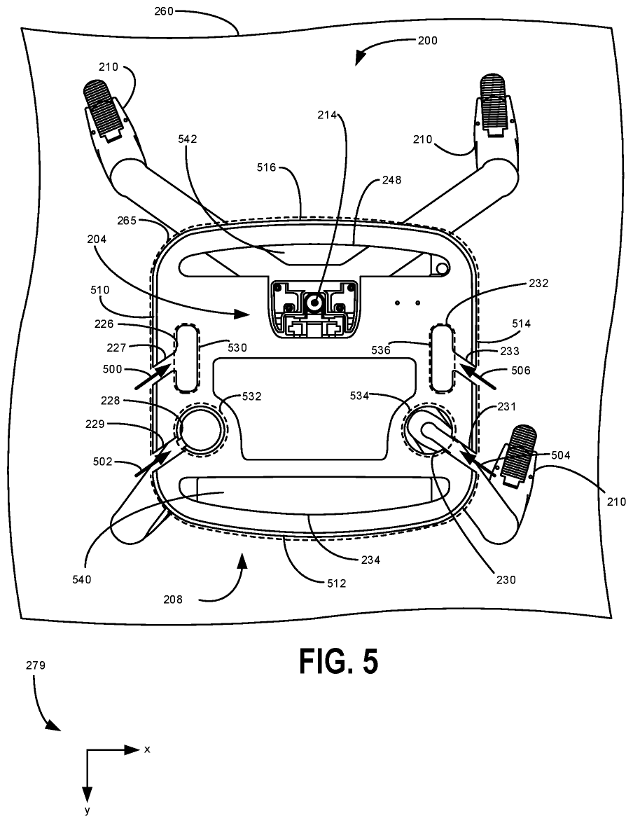

FIGS. 2-3 show different perspective views of an ultrasound imaging assembly 200 including a portable ultrasound system 202. FIG. 4 shows the portable ultrasound system 202 in a pivoted position relative to other components of the ultrasound imaging assembly 200, and FIG. 5 shows a top view of a tray 208 of the ultrasound imaging assembly 200 with a support stand 204 shown in cross-section. FIGS. 6-8 show different views of a cradle 206 of the ultrasound imaging assembly 200, and FIGS. 9-12 show different views of the portable ultrasound system 202. FIGS. 13-14 show views of an accessory support 1300 that may be coupled with the portable ultrasound system, and FIG. 15 shows a support arm 900 of the portable ultrasound system 202 in an exploded view. FIGS. 16-17 show different extended positions of the support arm 900, and FIGS. 18-21 show views of a cable support tab 1800 of the portable ultrasound system 202. Reference axes 279 are included by each of FIGS. 2-5 for comparison of the views shown.

Turning now to FIG. 2, a perspective view of the ultrasound imaging assembly 200 is shown. The ultrasound imaging assembly 200 includes portable ultrasound system 202. In one example, the portable ultrasound system 202 is similar to the system 100 described above with reference to FIG. 1. The portable ultrasound system 202 is a unitary system that is capable of being separated (e.g., decoupled) from a remainder of the ultrasound imaging assembly 200 and may be moved (e.g., portably) from room to room relative to the remainder of the ultrasound imaging assembly 200 which may stay in place and/or not be moved with the portable ultrasound system 202. The portable ultrasound system 202 may include several components similar to those described above with reference to system 100 (e.g., similar to display device 118, memory 120, system controller 116, probe 106, transmitter 102, receiver 108, etc.). The portable ultrasound system 202 is described in further detail below with reference to FIGS. 9-21.

As shown in the views shown by FIGS. 2-3, ultrasound imaging assembly 200 includes support stand 204, with tray 208, a storage container 212, and cradle 206 coupled to the support stand 204. Specifically, the tray 208 and storage container 212 are shown coupled to the support stand 204 between a first end 250 and a second end 252, with the cradle 206 positioned at the first end 250 and with a plurality of casters 210 of the support stand 204 positioned at the second end 252. Casters 210 are configured to support the support stand 204 against ground surface 260 and to enable the support stand 204 to more easily move across the ground surface 260 (e.g., roll along the ground surface 260). The z-axis of reference axes 279 is an axis positioned vertical relative to the ground surface 260 (e.g., extending in a vertical, normal direction relative to ground surface 260). In some examples, one or more of the casters 210 may be configured with a locking mechanism (e.g., a brake) configured to selectably lock the casters 210 and maintain a position of the support stand 204 relative to the ground surface 260 (e.g., reduce a likelihood of the casters 210 from rolling or otherwise moving relative to the ground surface 260).

In the views shown by FIGS. 2-3, storage container 212 is shown coupled to the support stand 204. Storage container 212 may be configured with a reservoir 262 adapted to store one or more accessories (e.g., fluid containers, disinfectants, etc.), such as accessory 243. Storage container 212 includes a first end 264 and a second end 266. In the example shown by FIGS. 2-3, second end 266 is closed (e.g., sealed) and first end 264 is open (e.g., not sealed) such that accessories may be inserted into the first end 264 in order to be stored within the reservoir 262 of the storage container 212. In other examples, the storage container 212 may include a different configuration. For example, the storage container 212 may be closed at each of the first end 264 and second end 266 and may include one or more slots adapted to enable an operator of the ultrasound imaging assembly 200 to access the reservoir 262 (e.g., for storage of accessories within the reservoir 262 and/or removal of accessories from the reservoir 262). In some examples, storage container 212 may include one or more grooves, tabs, etc. configured to engage with counterpart grooves, tabs, etc. of the support stand 204 in order to couple the storage container 212 to the support stand 204. For example, storage container 212 may include one or more tabs configured to engage with counterpart grooves of the support stand 204. In other examples, the storage container 212 may be coupled to the support stand 204 via one or more fasteners (e.g., bolts). Although storage container 212 is shown by FIGS. 2-3, in some examples the ultrasound imaging assembly 200 may not include the storage container 212.

FIGS. 2-3 additionally show the tray 208 coupled to the ultrasound imaging assembly 200. Tray 208 may include a plurality of openings adapted to maintain a position of one or more accessories of the ultrasound imaging assembly 200. For example, the plurality of openings of the tray 208 may be adapted to maintain a position of one or more probes, cables, etc. of the portable ultrasound system 202 during conditions in which the portable ultrasound system 202 is coupled to the support stand 204 via the cradle 206 (as described further below). As shown by FIGS. 2-5, tray 208 includes a first opening 226, a second opening 228, a third opening 230, and a fourth opening 232. First opening 226 is joined with a first slot 227, second opening 228 is joined with a second slot 229, third opening 230 is joined with a third slot 231, and fourth opening 232 is joined with a fourth slot 233. Each of the first opening 226, second opening 228, third opening 230, and fourth opening 232 are encircled by a perimeter 265 of the tray 208.

As shown by FIG. 5, tray 208 includes sidewall 510, sidewall 512, sidewall 514, and sidewall 516, with the sidewalls 510, 512, 514, and 516 together forming a perimeter 265 of the tray 208. Each of the slots described above (e.g., first slot 227, second slot 229, third slot 231, and fourth slot 233) extends outward to the perimeter 265 of the tray 208. Specifically, first slot 227 extends from first opening 226 to sidewall 510 and is open at sidewall 510, second slot 229 extends from second opening 228 to sidewall 510 and is open at sidewall 510, third slot 231 extends from third opening 230 to sidewall 514 and is open at sidewall 514, and fourth slot 233 extends from fourth opening 232 to sidewall 514 and is open at sidewall 514. In one example, each slot (e.g., first slot 227, second slot 229, third slot 231, and fourth slot 233) and each opening (e.g., first opening 226, second opening 228, third opening 230, and fourth opening 232) is shaped to maintain a position of cables of the portable ultrasound system 202 during conditions in which the portable ultrasound system 202 is coupled to the support stand 204.

For example, as shown by FIGS. 2-3, probes (e.g., probe 218, probe 220, etc.) and/or other devices coupled to the portable ultrasound system 202 (e.g., stored at a storage section 236 of the portable ultrasound system 202, described further below) may include a plurality of electrical cables 280 coupled (e.g., electrically connected) to one or more ports (e.g., data input and/or output ports) of the portable ultrasound system 202. During conditions in which the portable ultrasound system 202 is coupled to the support stand 204 (e.g., as shown by FIGS. 2-3), the electrical cables 280 may be inserted through one or more of the openings via the corresponding slots, and the position of the electrical cables 280 may be maintained relative to the support stand 204 by the openings of the tray 208. Example insertion directions of the cables 280 into the openings of the tray 208 are shown by FIG. 5. For example, arrow 500 indicates an example insertion direction of one or more of the cables 280 into the first opening 226 via first slot 227, arrow 502 indicates an example insertion direction of one or more of the cables 280 into the second opening 228 via second slot 229, arrow 504 indicates an example insertion direction of one or more of the cables 280 into the third opening 230 via third slot 231, and arrow 506 indicates an example insertion direction of one or more of the cables 280 into the fourth opening 232 via fourth slot 233. Specifically, the cables 280 may slide through the first slot 227 into the first opening 226 as indicated by arrow 500, cables 280 may slide through the second slot 229 into the second opening 228 as indicated by arrow 502, cables 280 may slide through the third slot 231 into the third opening 230 as indicated by arrow 504, and cables 280 may slide through the fourth slot 233 into the fourth opening 232 as indicated by arrow 506. By enabling the cables 280 to slide into the openings via the slots as described above, the position of the cables 280 relative to the tray 208 and the support stand 204 may be more easily maintained.

As shown by FIG. 4, the tray 208 includes a top surface 400 and a bottom surface 402. During conditions in which the tray 208 is coupled to the support stand 204 and the support stand 204 is in an upright position (e.g., a position in which the support stand 204 is supported by the ground surface 260 via casters 210), the bottom surface 402 is positioned closer to the ground surface 260 than the top surface 400. Each of the openings (e.g., first opening 226, second opening 228, third opening 230, and fourth opening 232) and each of the slots (e.g., first slot 227, second slot 229, third slot 231, and fourth slot 233) extends through an entire thickness of the tray 208 from the top surface 400 to the bottom surface 402.

Although the tray 208 is shown including four openings and four slots in FIGS. 2-5, in other examples the tray 208 may include a different number of openings and/or a different number of slots. Further, in some examples, one or more of the openings and/or slots may be shaped and/or positioned differently than the examples shown by FIGS. 2-5. For example, as shown by FIG. 5, the first opening 226 and fourth opening 232 each have a rounded, rectangular shape (e.g., cross-section) in a plane parallel to the top surface 400 and bottom surface 402 as indicated by outline 530 and outline 536 (respectively), and the second opening 228 and third opening 230 each have a circular shape (e.g., circular cross-section) in the plane parallel to the top surface 400 and bottom surface 402 as indicated by outline 532 and outline 534 (respectively). In other examples, one or more of the openings may be shaped differently relative to the outlines 530, 532, 534, and 536.

In some examples, one or more of the openings (e.g., first opening 226, second opening 228, third opening 230, and fourth opening 232) may be shaped to maintain a position of one or more probes, accessories, etc. of the ultrasound imaging assembly 200. For example, as shown by FIGS. 2-3, second opening 228 is shaped to maintain a position of accessory 219 relative to the tray 208 and support stand 204, and third opening 230 is shaped to maintain a position of probe 222 relative to the tray 208 and support stand 204. In one example, accessory 219 may be a bottle configured to store disinfectant, lubricant, etc. In other examples, accessory 219 may be a different type of accessory (e.g., accessory 219 may instead be a probe, similar to probe 222). A position of cables 280 coupled to the probe 222 may be maintained relative to the tray 208 and support stand 204 by fourth opening 232. In yet other examples, the openings of the tray 208 may be configured in a different way (e.g., with first opening 226, third opening 230, and fourth opening 232 each shaped to maintain a position of cables 280, and with second opening 228 shaped to maintain a position of a probe, such as probe 222).

Tray 208 further includes a first handle 234 and an opposing, second handle 248. First handle 234 forms a portion of first orifice 540, and second handle 248 forms a portion of second orifice 542, with the first orifice 540 being positioned opposite to the second orifice 542, and with the first handle 234 being positioned opposite to the second handle 248. Specifically, the first handle 234 forms the portion of the first orifice 540 and the sidewall 516, and the second handle 248 forms the portion of the second orifice 542 and the sidewall 512, with the sidewall 516 being opposite to the sidewall 512 (e.g., sidewall 516 and sidewall 512 form opposite ends of the perimeter 265). During conditions in which the tray 208 is coupled to the support stand 204, the second handle 248 is positioned closer to the support stand 204 than the first handle 234 (e.g., a length from the second handle 248 to the support stand 204 is less than a length from the first handle 234 to the support stand 204, in a direction parallel to the sidewalls 510 and 514 between the sidewalls 516 and 512). As shown by FIG. 5, the second orifice 542 encloses (e.g., encircles) a portion of the support stand 204 in the plane parallel to the top surface 400 and bottom surface 402 of the tray 208.

The tray 208 may include one or more features configured to enable the tray 208 to couple to the support stand 204. In some examples, tray 208 may include one or more grooves, tabs, etc. configured to engage with counterpart grooves, tabs, etc. of the support stand 204 in order to couple the tray 208 to the support stand 204. For example, tray 208 may include one or more tabs configured to engage with counterpart grooves of the support stand 204. In other examples, the tray 208 may be coupled to the support stand 204 via one or more fasteners (e.g., bolts). In some examples, the tray 208 may be slideable to a plurality of different positions along the support stand 204 (e.g., in a direction of axis 214) and may be maintained in any of the plurality of positions via the fasteners, tabs and grooves, etc. Although tray 208 is shown by FIGS. 2-5, in some examples the ultrasound imaging assembly 200 may not include the tray 208.

Support stand 204 is adjustable to a plurality of different heights. For example, the support stand 204 is shown by FIGS. 2-3 in a first configuration in which the support stand 204 has a first height 290 (e.g., a first length from the ground surface 260 to a pivot assembly 224 of the support stand 204, with the pivot assembly 224 positioned at the first end 250 of the support stand 204). FIG. 4 shows the support stand 204 in a second configuration in which the support stand has a second height 420 (e.g., a second length from the ground surface 260 to the pivot assembly 224). In the examples shown, the height of the support stand 204 in the first configuration (e.g., the first height 290 shown by FIGS. 2-3) is greater than the height of the support stand 204 in the second configuration (e.g., the second height 420 shown by FIG. 4).

The support stand 204 may include one or more locking mechanisms (e.g., locking pins, levers, etc.) configured to maintain the support stand 204 at a plurality of different heights (e.g., the first height 290, the second height 420, and a plurality of heights between the first height 290 and the second height 420), and the support stand 204 may be adjusted to (and/or locked at) any of the plurality of different heights by the operator of the ultrasound imaging assembly 200. By adjusting the height of the support stand 204, the operator may adjust the position of the pivot assembly 224 relative to the ground surface 260. For example, increasing the height of the support stand 204 may increase a length between the pivot assembly 224 and the ground surface 260, and decreasing the height of the support stand 204 may decrease the length between the pivot assembly 224 and the ground surface 260 (e.g., the length in the direction of axis 214, with axis 214 being parallel to the support stand 204 and normal relative to the ground surface 260). By increasing or decreasing the length between the pivot assembly 224 and the ground surface 260 via adjusting the height of the support stand 204 as described above, a height of the portable ultrasound system 202 relative to the ground surface 260 (e.g., a length between the portable ultrasound system 202 and the ground surface 260 in the direction of axis 214) may be adjusted during conditions in which the portable ultrasound system 202 is coupled to the support stand 204.

The cradle 206 of the ultrasound imaging assembly 200 is coupled to the support stand 204 via the pivot assembly 224. Pivot assembly 224 includes a plurality of pivots configured to enable the cradle 206 to pivot relative to the support stand 204. For example, as shown by FIG. 3, the pivot assembly 224 includes a first pivot 223 and a second pivot 225, with the first pivot 223 enabling the cradle 206 to pivot around axis 296, and with the second pivot 225 enabling the cradle 206 to pivot around axis 294. Axis 296 is parallel to axis 214 and the z-axis of reference axes 279, and axis 294 is perpendicular to axis 296 (e.g., axis 296 and axis 294 are perpendicular relative to each other). Specifically, the pivot assembly 224 includes an arm 297 extending between the first pivot 223 and the second pivot 225 and coupled to each of the first pivot 223 and the second pivot 225, with the first pivot 223 coupling the arm 297 to the support stand 204 and with the second pivot 225 coupling the arm 297 to a mounting bracket 298. The mounting bracket 298 is pivotable relative to the arm 297 via the second pivot 225, and the arm 297 is pivotable relative to the support stand 204 via the first pivot 223. The first pivot 223 enables the cradle 206 to pivot around axis 296 and does not enable the cradle 206 to pivot in other directions via the first pivot 223. The second pivot 225 enables the cradle 206 to pivot around axis 294 and does not enable the cradle 206 to pivot in other directions via the second pivot 225. However, in other examples, one or both of the first pivot 223 and second pivot 225 may be configured differently (e.g., as ball joints) in order to enable pivoting of the cradle with more than one degree of freedom per pivot.

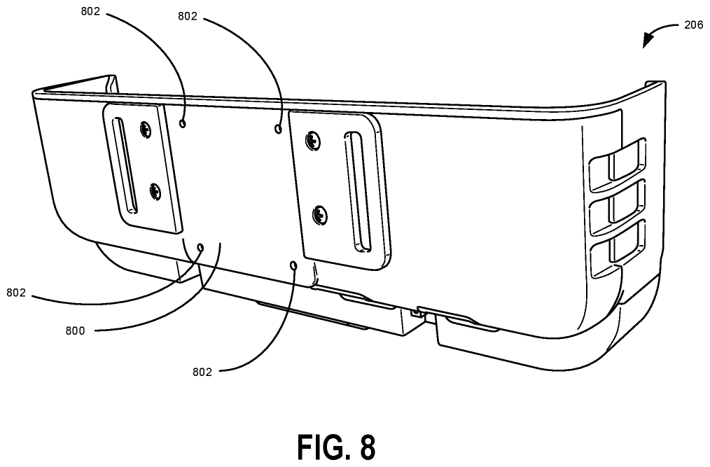

Mounting bracket 298 is fixedly coupled to cradle 206, such that the cradle 206 does not pivot relative to the mounting bracket 298 (or vice versa). In one example, the mounting bracket 298 may be coupled to the cradle 206 via one or more fasteners (e.g., bolts). For example, as shown by FIG. 8, the cradle 206 includes a mounting surface 800 having a plurality of openings 802 (e.g., holes). The plurality of openings 802 of the mounting surface 800 are positioned to align with corresponding, counterpart holes of the mounting bracket 298. Fasteners may be inserted through openings 802 of the mounting surface 800 and corresponding openings of the mounting bracket 298 in order to fixedly couple the cradle 206 to the mounting bracket 298. By fixedly coupling the mounting bracket 298 with the cradle 206, and by enabling the mounting bracket 298 to pivot around axis 294 via the second pivot 225, an angle of the portable ultrasound system 202 may be adjusted relative to the support stand 204 (and relative to the ground surface 260). For example, during conditions in which the portable ultrasound system 202 is coupled to the support stand 204 (e.g., by seating the portable ultrasound system 202 in the cradle 206 and locking the portable ultrasound system 202 to the cradle 206, as described below), the portable ultrasound system 202 may be pivoted toward and/or away from the support stand 204 and ground surface 260 via the mounting bracket 298 coupled to the second pivot 225 (e.g., as indicated by arrow 299 shown by FIG. 3). By pivoting the portable ultrasound system 202 around axis 294, the operator of the ultrasound imaging assembly 200 may adjust the portable ultrasound system 202 to be more easily viewable, in one example (e.g., to reduce light glare against the portable ultrasound system 202, etc.).

The mounting bracket 298 may include elements (e.g., biasing members, etc.) adapted to damp a vibration of the portable ultrasound system 202 during conditions in which the portable ultrasound system 202 is coupled to the cradle 206. For example, the portable ultrasound system 202 includes a touch-sensitive display 289 (e.g., shown in further detail by FIG. 9 and described below, and similar to the example described above with reference to the display device 118 shown FIG. 1), and because the operator of the ultrasound imaging assembly 200 may interact with the touch-sensitive display 289 via touch (e.g., as described above with reference to display device 118 of FIG. 1), the touch of the operator may result in forces against the portable ultrasound system 202. Touch-sensitive display 289 may be referred to herein as a touch-sensitive graphical display.

In order to reduce a likelihood of movement (e.g., pivoting) of the portable ultrasound system 202 in response to the touch of the operator of the ultrasound imaging assembly 200 during conditions in which the portable ultrasound system 202 is coupled to the cradle 206, the elements of the mounting bracket 298 adapted to dampen the vibration of the portable ultrasound system 202 may absorb (e.g., dampen) a portion of the forces applied to the portable ultrasound system 202 by the operator. For example, as the operator interacts with the portable ultrasound system 202 (e.g., moves a finger/glove/stylus across a face of the touch-sensitive display 289 in order to move a cursor atop an ultrasound image displayed by the touch-sensitive display 289 in a corresponding manner), one or more biasing members within the mounting bracket 298 may apply a restoring force to the portable ultrasound system 202 in order to counteract forces applied to the portable ultrasound system 202 that may alter a position of the portable ultrasound system 202 relative to the support stand 204 and ground surface 260. In this way, the operator of the ultrasound imaging assembly 200 may interact with the portable ultrasound system 202 during conditions in which the portable ultrasound system 202 is coupled to the cradle 206 (e.g., in order to manipulate an image shown by the portable ultrasound system 202, a graphical user interface of the portable ultrasound system 202, etc.) without altering the position of the portable ultrasound system 202 relative to the support stand 204 and ground surface 260.

The cradle 206 coupled to the mounting bracket 298 of the arm 297 of the pivot assembly 224 is rotatable (e.g., pivotable) in a plurality of directions via the first pivot 223 and second pivot 225. For example, the cradle 206 may pivot toward the ground surface 260 (e.g., in the direction of arrow 299) or away from the ground surface 260 (e.g., in the direction opposite to arrow 299) via the second pivot 225 (e.g., by pivoting the mounting bracket 298 relative to the arm 297 via the second pivot 225, with the mounting bracket 298 fixedly coupled to the cradle 206 such that the mounting bracket 298 and cradle 206 pivot together via the second pivot 225). Additionally, the cradle 206 may pivot around the support stand 204 and parallel to the ground surface 260 (e.g., in the direction of arrow 288, or the direction opposite to arrow 288) via the first pivot 223 (e.g., by pivoting the arm 297 relative to the support stand 204 via the first pivot 223).

Because the portable ultrasound system 202 may couple (e.g., lock) to the cradle 206, pivoting the cradle 206 as described above may pivot the portable ultrasound system 202 and adjust a position of the portable ultrasound system 202 relative to the support stand 204 and the ground surface 260. For example, FIGS. 2-3 show the cradle 206 (and portable ultrasound system 202) in a first position relative to the support stand 204 (e.g., a position in which length 285 of the cradle 206 is positioned perpendicular to the sidewall 510 and sidewall 514 of tray 208, with the sidewalls of the tray 208 indicated in FIG. 5), and FIG. 4 shows the cradle 206 in a second position relative to the support stand 204 (e.g., a position in which the length 285 of the cradle 206 is positioned at a different angle to the sidewall 510 and sidewall 514 relative to the first position, with the cradle 206 pivoted via the first pivot 223 in the direction opposite to arrow 288 around axis 296 relative to the first position).

FIGS. 6-7 show the cradle 206 removed from the mounting bracket 298 and with the portable ultrasound system 202 decoupled from the cradle 206. The cradle 206 includes a lock mechanism 602 adapted to lock the portable ultrasound system 202 to the cradle 206. In the example shown by FIGS. 6-7, the lock mechanism 602 includes a first hook 604 and a second hook 606. The first hook 604 and second hook 606 are shaped to couple with one or more counterpart openings (e.g., slots) included by the portable ultrasound system 202 (described in further detail below with reference to FIG. 12) in order to lock the portable ultrasound system 202 to the cradle 206 (e.g., maintain the portable ultrasound system 202 in a seated position against the cradle 206). Each of the first hook 604 and second hook 606 are coupled to an unlock mechanism 600. In the example shown by FIGS. 6-7, the unlock mechanism 600 includes a lever 601 positioned at a bottom end 610 of the cradle 206, with the bottom end 610 being closer to the ground surface 260 during conditions in which the cradle 206 is coupled to the mounting bracket 298 than a top end 608 of the cradle 206.

In order to couple the portable ultrasound system 202 to the cradle 206, the portable ultrasound system 202 may be inserted through the open, top end 608 of the cradle 206 and seated against a support surface 616 at the closed, bottom end 610 of the cradle 206. As the portable ultrasound system 202 is seated against the support surface 616, the first hook 604 and second hook 606 extending outward from the support surface 616 in the direction of the top end 608 are inserted into the one or more counterpart slots of the portable ultrasound system 202 and engage with the one or more counterpart slots in order to lock the portable ultrasound system 202 to the cradle 206. In one example, the first hook 604 and the second hook 606 may be biased by one or more biasing members in the direction of the top end 608 of the cradle 206 and a front end 624 of the cradle 206. As the first hook 604 and second hook 606 are inserted into the counterpart slots of the portable ultrasound system 202, the first hook 604 and second hook 606 may slide within corresponding slots of the support surface 616 (e.g., first slot 620 and second slot 622, respectively) in a direction from the front end 624 of the cradle 206 toward an opposing, rear end 626 of the cradle 206. Once the first hook 604 and second hook 606 are fully inserted into the counterpart slots of the portable ultrasound system 202, the first hook 604 and second hook 606 may slide in an opposite direction (e.g., a direction from the rear end 626 of the cradle 206 toward the front end 624 of the cradle 206) due to the biasing of the first hook 604 and second hook 606 toward the front end 624 by the one or more biasing members and lock into engagement with the counterpart slots of the portable ultrasound system 202. In this configuration, the portable ultrasound system 202 is locked to the cradle 206 via the first hook 604 and second hook 606 and cannot be removed from the cradle 206.

In order to decouple (e.g., unlock) the portable ultrasound system 202 from the cradle 206, an operator of the cradle 206 (e.g., the operator of the ultrasound imaging assembly 200 shown by FIGS. 2-4) may interact with the unlock mechanism 600 in order to release the lock mechanism 602 from engagement with the portable ultrasound system 202 (e.g., disengage the hooks of the lock mechanism from the corresponding openings of the portable ultrasound system 202). For example, the operator may apply force to the lever 601 (e.g., the operator may pull the lever 601) in an upward direction (e.g., a direction from the bottom end 610 of the cradle 206 toward the top end 608 of the cradle 206) in order to adjust a position of the first hook 604 and the second hook 606 relative to the support surface 616 and the portable ultrasound system 202. As the operator applies force to the lever 901 in the upward direction, the unlock mechanism 600 may adjust an amount of compression of the one or more biasing members (e.g., the one or more biasing members biasing the first hook 604 and the second hook 606 in the direction toward the front end 624 of the cradle 206) in order to move (e.g., slide) the first hook 604 and the second hook 606 in the direction away from the front end 624 and toward the rear end 626 (e.g., move the first hook 604 and the second hook 606 within the first slot 620 and the second slot 622, respectively). Moving the first hook 604 and the second hook 606 away from the front end 624 via the unlock mechanism 600 results in the first hook 604 and the second hook 606 disengaging from the counterpart slots of the portable ultrasound system 202 and unlocking the portable ultrasound system 202 from the cradle 206. The portable ultrasound system 202 may then be removed from the cradle 206 by the operator.

By configuring the portable ultrasound system 202 to lock and/or unlock with the cradle 206 via the lock mechanism 602 and unlock mechanism 600 (respectively), the portable ultrasound system 202 may be easily and quickly coupled and/or decoupled from the cradle 206 (e.g., for transporting the portable ultrasound system 202 to a different location than other components of the ultrasound imaging assembly 200, such as the support stand 204, tray 208, etc.). For example, during conditions in which it is desirable to use the portable ultrasound system 202 in a mounted state (e.g., coupled to the cradle 206 of the support stand 204), the portable ultrasound system 202 may be locked to the cradle 206 via the lock mechanism 602 as described above.

In one example, the operator may lock the portable ultrasound system 202 to the cradle 206 in order to enable the position of the portable ultrasound system 202 (e.g., the angle, vertical position, etc. of the portable ultrasound system 202 relative to the operator) to be more easily adjusted via the pivot assembly 224. In another example, the operator may lock the portable ultrasound system 202 to the cradle 206 in order to utilize cable management features of the ultrasound imaging assembly 200 (e.g., maintain the position of cables 280 within one or more openings of the tray 208, such as first opening 226, fourth opening 232, etc.), to more easily reach accessories of the ultrasound imaging assembly 200 (e.g., accessory 219, accessory 243, etc.) at a location proximate to the portable ultrasound system 202, etc.

In another example, the operator may unlock the portable ultrasound system 202 from the cradle 206 in order to move the portable ultrasound system 202 to a different location than the support stand 204. For example, the portable ultrasound system 202 may be unlocked (e.g., decoupled) from the cradle 206 via the unlock mechanism 600 and moved to a different room relative to a room in which the support stand 204 and cradle 206 are housed. Further, in some examples, the portable ultrasound system 202 may be coupled to a different cradle of a different support stand in the different room. Specifically, a first room may house the support stand 204 and cradle 206, and the portable ultrasound system 202 may initially be coupled to the cradle 206 in the first room. The operator of the ultrasound imaging assembly 200 may decouple the portable ultrasound system 202 from the cradle 206 and move the portable ultrasound system 202 to a different, second room including a second support stand having a second cradle, with the second support stand and second cradle being similar to the support stand 204 and cradle 206. The operator may then couple the portable ultrasound system 202 to the second cradle of the second support stand in order to utilize the portable ultrasound system 202 in a mounted configuration with the second cradle and second support stand in the second room. In this way, the portable ultrasound system 202 may be moved by the operator to a plurality of different locations (e.g., in order to image different patients, etc.) without moving the support stand 204, and a mobility (e.g., portability) of the portable ultrasound system 202 is increased. In some examples, as described below with reference to FIGS. 15-17, the portable ultrasound system 202 may include one or more support arms in order to enable a position of the portable ultrasound system 202 to be further adjusted during conditions in which the portable ultrasound system 202 is supported by surfaces not included by the cradle 206 (e.g., a tabletop).

Although the lock mechanism 602 and unlock mechanism 600 are described above independently, in some examples the lock mechanism 602 and unlock mechanism 600 may share one or more components and/or may be formed together as a single mechanism enabling both locking and unlocking of the portable ultrasound system 202 relative to the cradle 206. In some examples, components of the lock mechanism 602 and/or unlock mechanism may be shaped and/or positioned differently than the components described above and/or shown by FIGS. 6-7 (e.g., first hook 604, second hook 606, lever 601, etc.). For example, the lock mechanism 602 may include a different number of hooks (e.g., three hooks) and/or differently shaped hooks in some examples, the lever 601 may be shaped differently (e.g., shaped as a rod) and/or actuated in a different way (e.g., pushed, twisted, etc. by the operator), etc. Other variations are possible. However, in each example, the cradle 206 includes features shaped to engage with counterpart features of the portable ultrasound system 202 in order to lock and/or unlock the portable ultrasound system 202 with the cradle 206 (e.g., to enable the operator to easily seat and/or remove the portable ultrasound system 202 relative to the cradle 206 without tools or additional fasteners).

Turning now to FIGS. 9-12, various views of the portable ultrasound system 202 are shown, with the portable ultrasound system 202 decoupled (e.g., removed) from the cradle 206. As described above, the portable ultrasound system 202 includes the touch-sensitive display 289. The operator of the portable ultrasound system 202 may interact with the touch-sensitive display 289 (e.g., touch the touch-sensitive display with a finger, stylus, etc.) in order to manipulate images shown by the touch-sensitive display 289 and/or navigate a graphical user interface displayed by the touch-sensitive display 289. The touch-sensitive display 289 is housed by a housing 902 (which may be referred to herein as a casing), with the touch-sensitive display 289 being partially enclosed by the housing 902. Specifically, the touch-sensitive display 289 is partially enclosed by the housing 902 such that only a front end 908 of the touch-sensitive display 289 is visible at an exterior of the housing 902. For example, ends (or a perimeter) of the touch-sensitive display 289 may be enclosed and located interior to exterior surfaces of the housing 902 while the front end 908 of the touch-sensitive display 289, which includes a touchscreen portion of the display touchable via a user, is located exterior to the housing 902. The touch-sensitive display 289 may also be referred to herein as a touchscreen.

The housing 902 includes a first section 910 and a second section 912. The first section 910 and the second section 912 are joined together (e.g., formed as a single piece) so that they are integral with one another. The touch-sensitive display 289 is partially enclosed by the first section 910 as described above, and the second section 912 extends outward from the first section 910 at an angle relative to the touch-sensitive display 289 (e.g., as indicated in FIG. 9 by angle 918 between axis 916 extending parallel with the first section 910 and the touch-sensitive display 289 and axis 914 extending parallel with the second section 912). In the example shown by FIG. 9, the angle 918 is greater than ninety degrees (e.g., one-hundred-ten degrees), and thus is an obtuse angle, such that during conditions in which the portable ultrasound system 202 is coupled (e.g., locked) to the cradle 206 as described above and the second section 912 is positioned parallel with the ground surface 260 described above, the first section 910 (and touch-sensitive display 289) is angled relative to the ground surface 260. The first section 910 and second section 912 are angled relative to each other such that during conditions in which the portable ultrasound system 202 is coupled to the cradle 206, an angle of the second section 912 relative to the ground surface 260 is less than an angle of the first section 910 relative to the ground surface 260.

Angling the sections of the portable ultrasound system 202 as described above may increase an ease with which the operator may interact with the touch-sensitive display 289 (e.g., by positioning the touch-sensitive display 289 to angle away from the operator from the first section 910 to the second section 912, such that the touch-sensitive display 289 is tilted away from the operator). Further, angling the sections of the portable ultrasound system 202 as described above may increase an ability of the second section 912 to maintain a position of one or more probes and/or accessories stored by the second section 912, as described below. For example, angling the second section 912 relative to the first section 910 may enable a central axis of each of the one or more probes and/or accessories to be approximately parallel to a direction of gravity (e.g., a normal axis relative to the ground surface 260, such as axis 214 shown by FIGS. 2-5 and described above).

The second section 912 (which may be referred to herein as a probe storage section) includes a plurality of openings shaped to receive a plurality of probes and/or accessories. Specifically, the second section 912 includes opening 240, opening 242, opening 244, and opening 246, with each of the openings joined to an end 920 of the second section 912 and opened at the end 920. Each opening includes a corresponding sleeve shaped to maintain a position of probes and/or accessories seated within the openings. For example, as shown by FIG. 10, opening 240 is joined with sleeve 1001, opening 242 is joined with sleeve 1003, opening 244 is joined with sleeve 1005, and opening 246 is joined with sleeve 1006. Sleeves 1001, 1003, 1005, and 1006 may maintain a position of probes and/or accessories seated within the openings by partially enclosing the probes and/or accessories. Each of the sleeves 1001, 1003, 1005, and 1006 extend outward from the second section 912 in a direction toward rear end 909 of the portable ultrasound system 202 and may have a frustoconical shape tapering in diameter in the direction of the rear end 909 (e.g., having a smaller diameter toward the rear end 909 than toward the second section 912).

In the views shown by FIGS. 9-12, the portable ultrasound system 202 does not include the probes or accessories seated within the openings of the second section 912. However, FIGS. 2-3 show probe 220, probe 218, and accessory 216 coupled to the portable ultrasound system 202. Specifically, probe 220 may be seated within opening 246 and supported by sleeve 1006, probe 218 may be seated within opening 240 and supported by sleeve 1001, and accessory 216 may be seated within opening 242 and supported by sleeve 1003. In some examples, one or more of the sleeves (e.g., sleeve 1001, 1003, 1005, and 1006) may be removable from the portable ultrasound system 202 (e.g., for maintenance, replacement, etc.). Further, one or more of the sleeves may include openings adapted to receive electrical cables (e.g., cables 280) of the probes and/or accessories and to maintain a position of the electrical cables relative to the portable ultrasound system 202. By positioning the cables within the openings of the portable ultrasound system 202 (e.g., the openings of the sleeves as described above), the position of the cables relative to the portable ultrasound system 202 may be more easily maintained. For example, the operator may more easily move the portable ultrasound system 202 from one location to another (e.g., from the cradle 206 to a room that does not include the cradle 206 or support stand 204) by positioning the cables within the openings of the portable ultrasound system 202 in order to maintain the position of the cables.

An example of a sleeve that may be coupled with an opening (e.g., opening 242) of the housing 902 of the portable ultrasound system 202 is shown by FIGS. 13-14. Specifically, FIGS. 13-14 show accessory support 1300, with the accessory support 1300 including sleeve 1302 and cap 1304. In the configuration shown by FIG. 14, cap 1304 is inserted into the sleeve 1302 and forms a support surface of the sleeve 1302 for an accessory (e.g., accessory 216 shown by FIGS. 2-3).

Returning to FIGS. 9-12, the second section 912 further includes a handle 238 (which may be referred to herein as a carry handle). Handle 238 is formed by a central opening 930 of the second section 912, where the opening 930 is spaced away from end (e.g., end walls) of the second section 912. During conditions in which the operator desires to move the portable ultrasound system 202 (e.g., to a different location than the support stand 204, as described above), the operator may more easily grasp (e.g., hold) the portable ultrasound system 202 via the handle 238. For example, the operator may grasp the portable ultrasound system 202 with a first hand and decouple the portable ultrasound system 202 from the cradle 206 via a second hand (e.g., via the unlock mechanism 600 described above with reference to FIG. 6). In another example, during conditions in which one or more probes and/or accessories are coupled to (e.g., seated within) the openings of the second section 912, the operator may move (e.g., transport) the portable ultrasound system 202 by grasping the handle 238 in the first hand and grasping cables of the one or more probes and/or accessories (e.g., cables 280 described above) in the second hand, with the cables further maintained in position relative to the portable ultrasound system 202 by the plurality of openings.

As described herein, the term “portable” refers to the ability of the portable ultrasound system 202 to be easily transported from one location to another without the other components of the ultrasound imaging assembly 200 (e.g., support stand 204, cradle 206, tray 208, etc.). The handle 238 increases the portability of the portable ultrasound system 202 by enabling the portable ultrasound system 202 to be more easily carried by the operator.

As shown by FIG. 10, the portable ultrasound system 202 includes a plurality of electronic communication ports (which may be referred to herein as electronic input connections) adapted to electrically couple a controller of the portable ultrasound system 202 to one or more electronic devices (e.g., probes). In the example shown, the portable ultrasound system 202 includes port 1000, port 1002, and port 1004. In one example, probe 218, probe 220, and/or probe 222 shown by FIGS. 2-3 may be electronically (e.g., electrically) coupled to the portable ultrasound system 202 (e.g., the electronic controller of the portable ultrasound system 202 disposed within and enclosed by the housing 902 of the portable ultrasound system 202 and in electronic communication with the touch-sensitive display 289, similar to controller 116 shown by FIG. 1 and described above) via the ports described above. For example, probe 218 may be electrically coupled to port 1000 via a first cable (e.g., one of cables 280 shown by FIGS. 2-3 and described above), probe 220 may be electrically coupled to port 1002 via a different, second cable, and probe 222 may be electrically coupled to port 1004 via a different, third cable. Electronically coupling the probes to the portable ultrasound system 202 via the ports may enable the portable ultrasound system 202 to receive electrical signals from the probes (e.g., in order to image a patient).

Similar to the example of the controller 116 described above with reference to FIG. 1, the controller of the portable ultrasound system 202 includes computer readable instructions stored on non-transitory memory that when executed, cause the controller to acquire ultrasound data via one or more ultrasound probes (e.g., probe 218, probe 220, etc.), generate an image from the acquired ultrasound data, and display the image via the touch-sensitive display 289.

The portable ultrasound system 202 may include a battery disposed within the housing 902, with the battery electrically coupled to the controller and the electronic input connections described above (e.g., ports 1000, 1002, and 1004). The battery may provide electrical power to the portable ultrasound system 202 during conditions in which the portable ultrasound system 202 is decoupled from the cradle 206, for example (e.g., moved to a different location than the support stand 204). However, the battery may also provide electrical power to the portable ultrasound system 202 (e.g., to the controller and electronic input connections) during conditions in which the portable ultrasound system 202 is coupled to the cradle 206 (e.g., conditions in which the portable ultrasound system 202 is seated against the cradle 206 and is not coupled to an external power source, such as an electrical outlet of a wall). The portable ultrasound system may additionally include other electronic input connections (e.g., input connections 1050) adapted to couple the controller to other devices (e.g., electronic storage devices, such as thumb drives having non-transitory computer memory) and/or external power sources.

As shown by FIG. 9, the portable ultrasound system 202 may additionally include an indicator light array 904. The indicator light array 904 is in electronic communication with the controller and a status operation mode of the indicator light array 904 may be adjusted by the operator. For example, the indicator light array 904 may be adjustable to a plurality of lighting modes by the operator via the controller, where each lighting mode of the plurality of lighting modes corresponds to a different wavelength of light emitted by the indicator light array 904. The operator may interact with the touch-sensitive display 289 in order to select a lighting mode of the indicator light array 904 (e.g., via a graphical user interface of the portable ultrasound system 202). In one example, in a first lighting mode, the indicator light array 904 may emit light having a wavelength of approximately 500 nanometers, and in a second lighting mode, the indicator light array 904 may emit light having a wavelength of approximately 600 nanometers. Each lighting mode may correspond to a different wavelength of light emitted by the indicator light array 904 within a plurality of wavelengths (e.g., a range of wavelengths, such as 400 nanometers to 700 nanometers, corresponding to a visible spectrum of light). In one example, the operator may select a wavelength of light to be emitted by the indicator light array 904 (e.g., a color of light to be emitted by the indicator light array 904) in order to visually indicate that the portable ultrasound system 202 is assigned to the operator (e.g., the operator is the user of the portable ultrasound system 202). For example, the indicator light array 904 may emit blue light in order to indicate to others (e.g., other people, different than the operator) that the portable ultrasound system 202 is utilized by the operator. During conditions in which two or more portable ultrasound systems 202 are within a same facility (e.g., a same room, hospital, etc.), the indicator light array 904 of each portable ultrasound system 202 may emit a different color light relative to each other portable ultrasound system 202 in order to indicate which portable ultrasound system 202 is assigned to each operator. Other examples are possible.

In one example, the indicator light array 904 may emit different colors of light in response to an energization level of the battery disposed within the housing 902. For example, during conditions in which an amount of electrical energy (e.g., electrical charge) stored by the battery is greater than 50% of a maximum amount of electrical energy of the battery, the indicator light array 904 may emit a green color light (e.g., light having a wavelength of 530 nanometers), and during conditions in which the amount of electrical energy stored by the battery is less than 50% of the maximum amount, the indicator light array 904 may emit light having a different color than green (e.g., yellow light having a wavelength of 580 nanometers while the electrical energy is between 20% and 50% of the maximum, red light having a wavelength of 680 nanometers while the electrical energy is lower than 20% of the maximum, etc.).

In another example, the indicator light array 904 may produce light in a plurality of different patterns or sequences. For example, in various lighting modes, the indicator light array 904 may flash (e.g., blink) at one or more different frequencies. For example, in a first lighting mode, the indicator light array 904 may produce a flash of light once per second, with the flash having a duration of less than one second (e.g., one-half second). In a second lighting mode, the indicator light array 904 may produce a flash of light once per two seconds, with the flash having a duration of less than two seconds (e.g., one second). In a third lighting mode, the indicator light array 904 may produce two flashes of light per second, with each flash having a duration of less than one-half second. Further, in some examples, the flashes of light emitted by the indicator light array 904 may be colored differently relative to each other (e.g., each flash of light may correspond to a different wavelength of light). For example, in a fourth lighting mode, the indicator light array may produce two flashes of light per second, with each flash having a duration of less than one-half second, and with the first flash having a first color (e.g., a red color corresponding to a wavelength of the light being approximately 680 nanometers), and with the second flash having a different, second color (e.g., a yellow color corresponding to a wavelength of the light being approximately 580 nanometers). In one example, the various lighting modes of the indicator light array 904 may indicate an operating mode of the portable ultrasound system 202 (e.g., whether the portable ultrasound system 202 is idle or is being used to image a patient). In another example, the various lighting modes may indicate that the portable ultrasound system 202 is utilized by a particular operator, as described above. Other examples are possible.

FIG. 12 shows a bottom view of the portable ultrasound system 202 (e.g., a view of bottom end 940 of the portable ultrasound system 202 as indicated in FIGS. 9-10). During conditions in which the portable ultrasound system 202 is coupled to the cradle 206 (e.g., as shown by FIGS. 2-4), the bottom end 940 of the portable ultrasound system 202 is seated against the support surface 616 of the cradle 206 (shown by FIGS. 6-7) and is partially enclosed by the cradle 206 (e.g., surrounded by surfaces of the cradle 206). The bottom end 940 includes a first lock orifice 1200 and a second lock orifice 1202. First lock orifice 1200 and second lock orifice 1202 may be referred to herein as mating features and/or openings of the portable ultrasound system 202. The first lock orifice 1200 and the second lock orifice 1202 are shaped to engage with the hooks of the cradle 206 (e.g., first hook 604 and second hook 606, respectively, shown by FIGS. 6-7, which may be referred to herein as counterpart mating features of the cradle 206) in order to removably couple the housing 902 of the portable ultrasound system 202 to the cradle 206. Specifically, the hooks of the cradle 206 lock the portable ultrasound system 202 to the cradle 206 by engaging in face sharing contact with the corresponding lock orifices (e.g., first lock orifice 1200 and second lock orifice 1202, respectively). As such, the first lock orifice 1200 is positioned to align with the first hook 604 during conditions in which the portable ultrasound system 202 is seated against the support surface 616 of the cradle 206, and the second lock orifice 1202 is positioned to align with the second hook 606 during conditions in which the portable ultrasound system 202 is seated against the support surface 616 of the cradle 206. A length 790 between the first hook 604 and the second hook 606 (as shown by FIG. 7) may be approximately a same length as a length 1210 between the first lock orifice 1200 and the second lock orifice 1202 (as shown by FIG. 12). In the example shown, the first lock orifice 1200 and the second lock orifice 1202 are open at one end in order to enable insertion of the corresponding hooks of the cradle into the lock orifices. In other examples, the first lock orifice 1200 and/or the second lock orifice 1202 may be shaped differently.

FIGS. 9-10 and FIG. 12 additionally show support arm 900 coupled to the housing 902 of the portable ultrasound system 202 in a retracted position (e.g., a position in which a distal end 990 of the support arm 900 is engaged with, or positioned against, the housing 902). In some examples, the support arm 900 may be maintained in the retracted position by one or more lock mechanisms. For example, the support arm 900 may be maintained in the retracted position by a clip, latch, etc., and may be moved from the retracted position when the operator disengages the clip, latch, etc. from the support arm 900. In other examples, the support arm 900 may be maintained in the retracted position by friction, and may be moved from the retracted position by the operator.

The support arm 900 is shown in further detail in an exploded view relative to the housing 902 by FIG. 15. In the example shown, the support arm 900 includes a first extension 1506 and a parallel, second extension 1508 joined by a shaft 1510 extending in a perpendicular direction between the first extension 1506 and second extension 1508. In other examples, the support arm 900 may include a different relative arrangement of extensions and/or a different number of extensions. For example, first extension 1506 and second extension 1508 may be at an angle relative to each other (e.g., angled toward each other or away from each other). Further, in some examples, support arm 900 may be shaped differently (e.g., one or more of the first extension 1506, second extension 1508, and/or shaft 1510 may be shaped differently relative to the example shown by FIG. 15).

The support arm 900 is pivotally coupled to the housing 902 of the portable ultrasound system 202 via a plurality of pivots at an opposite end of the housing 902 relative to the touch-sensitive display 289 (e.g., positioned at rear end 909 shown by FIG. 9, opposite to front end 908). For example, as shown by FIG. 15, the support arm 900 is coupled to the housing 902 by a first hinge 1500 and a second hinge 1504. First hinge 1500 and second hinge 1504 may be referred to herein as pivots. The first hinge 1500 and second hinge 1504 are engaged with the support arm 900 to selectably maintain the support arm 900 in at least one extended position of a plurality of extended positions relative to the housing 902. The first hinge 1500 and the second hinge 1504 are pivotable relative to the housing (e.g., first hinge 1500 and second hinge 1504 are coupled to the housing 902 in such a way that the hinges may rotate relative to the housing 902). The support arm 900 is coupled to the housing 902 via the hinges such that the support arm 900 may rotate around axis 1512 via first hinge 1500 and second hinge 1504. In some examples, the support arm 900 may be coupled to the hinges and the housing 902 via one or more fasteners (e.g., bolts). In other examples, the support arm 900 may be coupled to the hinges and the housing 902 via one or more caps, such as cap 1502. In yet other examples, the support arm 900 may be coupled to the hinges and the housing 902 via a combination of caps and fasteners. Example positions of the support arm 900 relative to the housing 902 are described below with reference to FIGS. 16-17.

FIG. 16 shows a side view of the portable ultrasound system 202 with the support arm 900 in a first extended position. In the example shown by FIG. 16, the portable ultrasound system 202 is decoupled (e.g., removed) from the cradle 206 (shown by FIGS. 2-8) and is positioned on a support surface 1600 (e.g., a horizontal support surface relative to the ground surface 260 shown by FIGS. 2-5). Support surface 1600 is a surface separate from the ultrasound imaging assembly 200 (e.g., a surface not included by the components of the ultrasound imaging assembly 200) and may be an approximately flat, planar surface (e.g., a surface without curvature). For example, support surface 1600 may be a top surface of a table (e.g., a tabletop). The support surface 1600 is positioned horizontally, such that the axis 214 shown by FIGS. 2-5 is normal relative to the support surface 1600. However, in other examples, the support surface 1600 may be at a slight angle relative to the ground surface 260 (e.g., at an angle of 5° relative to the ground surface 260). Additionally, the support surface 1600 may not be located in a same location as the support stand 204 and cradle 206, in some examples. For example, the support surface 1600 may be a top surface of a table positioned in a separate room relative to the support stand 204 and cradle 206. As described above, the portable ultrasound system 202 may be decoupled from the cradle 206 and moved to locations exterior to the cradle 206. In one example, the operator may decouple the portable ultrasound system 202 from the cradle 206 and move the portable ultrasound system 202 into the room containing the table having the support surface 1600, with the table being in the separate, different room relative to the support stand 204 and cradle 206. The operator may then move the support arm 900 of the portable ultrasound system 202 to the extended position shown by FIG. 16 in order to position the portable ultrasound system 202 upright relative to the support surface 1600.

During conditions in which the support arm 900 is in the extended position shown by FIG. 16, the support arm 900 is angled relative to the housing 902 of the portable ultrasound system 202 by a first angle 1610. However, it should be noted that the extended position of the support arm 900 shown by FIG. 16 is one of a plurality of possible extended positions of the support arm 900 relative to the housing 902. A second extended position as shown by FIG. 17. In the example shown by FIG. 17, the support arm 900 is in a second extended position, with the support arm 900 being angled relative to the housing 902 by a second angle 1612. The second angle 1612 is a smaller amount of angle than the first angle 1610. In each extended position of the plurality of extended positions, the distal end 990 of the support arm 900 is disengaged with the housing 902 (e.g., not positioned in face-sharing contact with the housing 902 and spaced away from the housing 902). The support arm 900 may be movable into one of the plurality of extended positions only while the portable ultrasound system 202 is decoupled from the cradle 206 (e.g., while the distal end 990 of the support arm 900 is not positioned between the housing 902 and surfaces of the cradle 206 and in contact with surfaces of the housing 902 and cradle 206).

The support arm 900 is coupled to the housing 902 (e.g., via the hinges 1500 and 1504) in such a way that the support arm 900 may be moved to any of a plurality of different extended positions by the operator, with the selected extended position of the support arm 900 being maintained by the coupling between the support arm 900 and the housing 902 (e.g., the hinges). For example, the hinges 1500 and 1504 may be configured to maintain the support arm 900 in the selected extended position by reducing a likelihood of the support arm 900 pivoting in response to a weight of the portable ultrasound system 202 during conditions in which the portable ultrasound system 202 is not moved by the operator (e.g., conditions in which the operator is not applying force to the support arm 900). The hinges 1500 and 1504 may maintain the support arm 900 in the selected extended position until the operator applies force to the support arm 900 (e.g., in order to adjust the support arm 900 to a different extended position). In one example, the hinges 1500 and 1504 may be configured to maintain the position of the support arm 900 during conditions in which the operator is interacting with the touch-sensitive display 289 of the portable ultrasound system 202 while the portable ultrasound system 202 is positioned on the support surface 1600. The support arm 900 may resist rotation in response to force applied to the touch-sensitive display 289 by the operator in order to interact with the touch-sensitive display 289 (e.g., manipulate images and/or a graphical user interface displayed by the touch-sensitive display 209). However, the support arm 900 may rotate in response to the operator directly applying force to the support arm 900 in order to rotate support arm 900 (e.g., to one of the plurality of extended positions or the retracted position).

Turning now to FIGS. 18-21, cable support tab 1800 of the portable ultrasound system 202 is shown. As shown by FIG. 18, cable support tab 1800 couples with the portable ultrasound system 202 through a slot 1802 of the cradle 206. In one example, the portable ultrasound system 202 may include a slot (e.g., an opening in housing 902) shaped to receive a protrusion 1804 of the cable support tab 1800 (with the protrusion 1804 shown by FIG. 19). Cable support tab 1800 may be formed of a flexible material (e.g., rubber), and during conditions in which the cable support tab 1800 is inserted through the slot 1802 of the cradle 206 in order to couple with the portable ultrasound system 202, protrusion 1804 may temporarily deforms (e.g., compress) in order to fit within the slot of the housing 902 of the portable ultrasound system 202. Once the protrusion 1804 is fully inserted into the slot of the housing 902, the protrusion 1804 may return to its original, non-deformed shape in order to securely couple with the slot of the housing 902.

The cable support tab 1800 includes an angled extension 1806 and a groove 1808. One or more electrical cables 280 coupled to the portable ultrasound system 202 may be supported by the groove 1808 of the cable support tab 1800 (e.g., maintained in position relative to the portable ultrasound system 202 by a seating within the groove 1808). Because the cable support tab 1800 is formed from the flexible material, cable support tab 1800 may deform (e.g., bend, twist, etc.) without degradation. FIG. 20 shows the cable support tab 1800 in an uncompressed condition (e.g., a condition in which the cable support tab 1800 is not deformed), and FIG. 21 shows the cable support tab 1800 in a compressed condition (e.g., a condition in which the cable support tab 1800 is deformed as a result of one or more external forces applied to the cable support tab 1800), with FIG. 21 showing the uncompressed condition in dotted lines. The cable support tab 1800 may temporarily deform in response to the one or more external forces (e.g., pressing forces by the operator), and may return to the uncompressed condition when the one or more external forces are no longer applied to the cable support tab 1800. By enabling the cable support tab 1800 to deform, a likelihood of degradation of the cable support tab 1800 may be reduced. Although the cable support tab 1800 is described herein with reference to FIGS. 18-21, in some examples, the portable ultrasound system 202 and the cradle 206 may not include the cable support tab 1800.

FIGS. 2-21 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

By pivotally coupling the support arm to the housing of the portable ultrasound system, the portable ultrasound system may be supported on a plurality of different surfaces. The operator may decouple the portable ultrasound system from the cradle and move the portable ultrasound system to a different location via the carry handle, and the support stand may be pivoted to any of the extended positions in order to enable the operator to more easily interact with the touch-sensitive display of the portable ultrasound system. During conditions in which coupling the portable ultrasound system to the support stand is desired, the operator may seat the portable ultrasound system against the cradle of the support stand and engage the portable ultrasound system with the lock mechanism of the cradle to lock the portable ultrasound system to the cradle. Further, the probe storage section of the portable ultrasound system may maintain a position of one or more probes of the portable ultrasound system while the portable ultrasound system is moved from the support stand and/or coupled or decoupled from the cradle. In this way, the portable ultrasound system may be easily utilized in a larger variety of different locations, and the portability of the portable ultrasound system is increased.

The technical effect of configuring the lock mechanism of the cradle to engage with counterpart features of the portable ultrasound system is to enable the portable ultrasound system to lock to the cradle.

In one embodiment, a portable ultrasound imaging system comprises: a touch-sensitive graphical display; a controller in electronic communication with the display; and a housing enclosing the display and the controller, the housing including: an electronic input connection adapted to electrically couple the controller with an ultrasound probe; a probe storage section having a first opening shaped to receive the ultrasound probe; and a first mating feature adapted to engage with a counterpart, second mating feature of a cradle of a support stand to removably couple the housing to the cradle. In a first example of the portable ultrasound imaging system, the portable ultrasound imaging system further comprises a support arm pivotally coupled to the housing. A second example of the portable ultrasound imaging system optionally includes the first example, and further includes wherein the support arm is positioned at an opposite end of the housing relative to the touch-sensitive graphical display. A third example of the portable ultrasound imaging system optionally includes one or both of the first and second examples, and further includes wherein the support arm comprises a first extension and a parallel, second extension joined by a shaft extending in a perpendicular direction between the first extension and second extension. A fourth example of the portable ultrasound imaging system optionally includes one or more or each of the first through third examples, and further includes wherein the support arm is coupled to the housing via a pivot, the pivot engaged with the support arm to selectably maintain the support arm in at least one extended position of a plurality of extended positions relative to the housing, where the shaft is spaced away from the housing when the support arm is in the at least one extended position. A fifth example of the portable ultrasound imaging system optionally includes one or more or each of the first through fourth examples, and further includes wherein the probe storage section of the housing includes a second opening forming a carry handle. A sixth example of the portable ultrasound imaging system optionally includes one or more or each of the first through fifth examples, and further includes a battery disposed within the housing, the battery electrically coupled to the controller and the electronic input connection. A seventh example of the portable ultrasound imaging system optionally includes one or more or each of the first through sixth examples, and further includes wherein the touch-sensitive graphical display includes a display screen and an indicator light array, the indicator light array in electronic communication with the controller and adjustable to a plurality of lighting modes via the controller, where each lighting mode of the plurality of lighting modes corresponds to a different wavelength of light emitted by the indicator light. An eighth example of the portable ultrasound imaging system optionally includes one or more or each of the first through seventh examples, and further includes wherein the controller includes computer readable instructions stored on non-transitory memory that when executed, cause the controller to: acquire ultrasound data via the ultrasound probe; generate an image from the acquired ultrasound data; and display the image via the touch-sensitive graphical display.

In one embodiment, an ultrasound imaging assembly comprises: a portable ultrasound system including a touchscreen housed in a casing, the casing having an opening at a bottom end; and a cradle shaped to partially enclose the bottom end of the casing, the cradle including a lock mechanism adapted to engage with the opening at the bottom end of the casing to lock the portable ultrasound system to the cradle. In a first example of the ultrasound imaging assembly, the portable ultrasound system further includes a support arm, the support arm pivotable to a plurality of extended positions relative to the casing, the support arm adapted to engage with a horizontal support surface. A second example of the ultrasound imaging assembly optionally includes the first example, and further includes wherein the cradle further comprises an unlock mechanism adapted to disengage the lock mechanism from the bottom end of the casing. A third example of the ultrasound imaging assembly optionally includes one or both of the first and second examples, and further includes wherein the lock mechanism includes a hook shaped to engage with the opening, and wherein the unlock mechanism includes a lever coupled to the hook, the lever adapted to disengage the hook from the opening. A fourth example of the ultrasound imaging assembly optionally includes one or more or each of the first through third examples, and further includes wherein the cradle includes a mounting surface having a plurality of holes, with each hole of the plurality of holes positioned to align with a corresponding, counterpart hole of a mounting bracket of a support stand. A fifth example of the ultrasound imaging assembly optionally includes one or more or each of the first through fourth examples, and further includes wherein the cradle includes a slot positioned to align with a corresponding, counterpart slot of the casing, and further comprising a cable support tab shaped to removably couple with the slot of the cradle through the counterpart slot of the casing.

In another embodiment, an ultrasound imaging assembly comprises: a support stand; a cradle pivotally coupled to the support stand, the cradle including a lock mechanism; and a portable ultrasound system removably coupled to the cradle, the portable ultrasound system including a touch-sensitive display and a housing partially enclosing the display, the housing shaped to seat against a support surface of the cradle and engage with the lock mechanism of the cradle. In a first example of the ultrasound imaging assembly, the portable ultrasound system is removably coupleable to the cradle via the lock mechanism, and further comprising a support arm pivotally coupled to the housing of the portable ultrasound system, the support arm pivotable from a retracted position where a distal end of the support arm is engaged with the housing to a plurality of extended positions where the distal end is disengaged with the housing, the distal end arranged opposite to a pivot end of the support arm that is pivotably coupled to the housing, where the support arm is movable into one of the plurality of extended positions only while the portable ultrasound system is decoupled from the cradle. A second example of the ultrasound imaging assembly optionally includes the first example, and further includes wherein the cradle is pivotally coupled to the support stand by a pivot assembly, the pivot assembly including an arm coupled to the support stand by a first pivot and a mounting bracket coupled to the arm by a second pivot, the arm pivotable in a first direction via the first pivot and the mounting bracket pivotable in a different, second direction via the second pivot, with the mounting bracket fixedly coupled to the cradle. A third example of the ultrasound imaging assembly optionally includes one or both of the first and second examples, and further includes wherein the lock mechanism includes a plurality of hooks shaped to engage with a plurality of counterpart slots of the housing of the portable ultrasound system. A fourth example of the ultrasound imaging assembly optionally includes one or more or each of the first through third examples, and further includes wherein a length of the support stand in a vertical direction relative to a ground surface on which the support stand sits is adjustable to a plurality of different lengths.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A portable ultrasound imaging system, comprising:
a touch-sensitive graphical display;
a controller in electronic communication with the display; and
a housing enclosing the display and the controller, the housing including:
an electronic input connection adapted to electrically couple the controller with an ultrasound probe;
a probe storage section having a first opening shaped to receive the ultrasound probe; and
a first mating feature adapted to engage with a counterpart, second mating feature of a cradle of a support stand to removably couple the housing to the cradle.

2. The portable ultrasound imaging system of claim 1, further comprising a support arm pivotally coupled to the housing.

3. The portable ultrasound imaging system of claim 2, wherein the support arm is positioned at an opposite end of the housing relative to the touch-sensitive graphical display.

4. The portable ultrasound imaging system of claim 2, wherein the support arm comprises a first extension and a parallel, second extension joined by a shaft extending in a perpendicular direction between the first extension and second extension.

5. The portable ultrasound imaging system of claim 2, wherein the support arm is coupled to the housing via a pivot, the pivot engaged with the support arm to selectably maintain the support arm in at least one extended position of a plurality of extended positions relative to the housing, where the shaft is spaced away from the housing when the support arm is in the at least one extended position.

6. The portable ultrasound imaging system of claim 1, wherein the probe storage section of the housing includes a second opening forming a carry handle.

7. The portable ultrasound imaging system of claim 1, further comprising a battery disposed within the housing, the battery electrically coupled to the controller and the electronic input connection.

8. The portable ultrasound imaging system of claim 1, wherein the touch-sensitive graphical display includes a display screen and an indicator light array, the indicator light array in electronic communication with the controller and adjustable to a plurality of lighting modes via the controller, where each lighting mode of the plurality of lighting modes corresponds to a different wavelength of light emitted by the indicator light.

9. The portable ultrasound imaging system of claim 1, wherein the controller includes computer readable instructions stored on non-transitory memory that when executed, cause the controller to:
acquire ultrasound data via the ultrasound probe;
generate an image from the acquired ultrasound data; and
display the image via the touch-sensitive graphical display.

10. An ultrasound imaging assembly, comprising:
a portable ultrasound system including a touchscreen housed in a casing, the casing having an opening at a bottom end; and
a cradle shaped to partially enclose the bottom end of the casing, the cradle including a lock mechanism adapted to engage with the opening at the bottom end of the casing to lock the portable ultrasound system to the cradle.

11. The ultrasound imaging assembly of claim 10, wherein the portable ultrasound system further includes a support arm, the support arm pivotable to a plurality of extended positions relative to the casing, the support arm adapted to engage with a horizontal support surface.

12. The ultrasound imaging assembly of claim 10, wherein the cradle further comprises an unlock mechanism adapted to disengage the lock mechanism from the bottom end of the casing.

13. The ultrasound imaging assembly of claim 12, wherein the lock mechanism includes a hook shaped to engage with the opening, and wherein the unlock mechanism includes a lever coupled to the hook, the lever adapted to disengage the hook from the opening.

14. The ultrasound imaging assembly of claim 10, wherein the cradle includes a mounting surface having a plurality of holes, with each hole of the plurality of holes positioned to align with a corresponding, counterpart hole of a mounting bracket of a support stand.

15. The ultrasound imaging assembly of claim 10, wherein the cradle includes a slot positioned to align with a corresponding, counterpart slot of the casing, and further comprising a cable support tab shaped to removably couple with the slot of the cradle through the counterpart slot of the casing.

16. An ultrasound imaging assembly, comprising:
a support stand;
a cradle pivotally coupled to the support stand, the cradle including a lock mechanism; and
a portable ultrasound system removably coupled to the cradle, the portable ultrasound system including a touch-sensitive display and a housing partially enclosing the display, the housing shaped to seat against a support surface of the cradle and engage with the lock mechanism of the cradle.

17. The ultrasound imaging assembly of claim 16, wherein the portable ultrasound system is removably coupleable to the cradle via the lock mechanism, and further comprising a support arm pivotally coupled to the housing of the portable ultrasound system, the support arm pivotable from a retracted position where a distal end of the support arm is engaged with the housing to a plurality of extended positions where the distal end is disengaged with the housing, the distal end arranged opposite to a pivot end of the support arm that is pivotably coupled to the housing, where the support arm is movable into one of the plurality of extended positions only while the portable ultrasound system is decoupled from the cradle.

18. The ultrasound imaging assembly of claim 16, wherein the cradle is pivotally coupled to the support stand by a pivot assembly, the pivot assembly including an arm coupled to the support stand by a first pivot and a mounting bracket coupled to the arm by a second pivot, the arm pivotable in a first direction via the first pivot and the mounting bracket pivotable in a different, second direction via the second pivot, with the mounting bracket fixedly coupled to the cradle.

19. The ultrasound imaging assembly of claim 16, wherein the lock mechanism includes a plurality of hooks shaped to engage with a plurality of counterpart slots of the housing of the portable ultrasound system.

20. The ultrasound imaging assembly of claim 16, wherein a length of the support stand in a vertical direction relative to a ground surface on which the support stand sits is adjustable to a plurality of different lengths.
